(19) United States
(12) Patent Application Publication
(10) Pub. No.: US 2005/0118602 A1
(43) Pub. Date: Jun. 2, 2005

(54) Gene profiling of single or multiple cells

(76) Inventors: Yin-Xiong Li, Durham, NC (US); Margaret L. Kirby, Durham, NC (US)

Correspondence Address: MYERS BIGEL SIBLEY & SAJOVEC, PO BOX 37428, RALEIGH, NC, 27627, US

(21) Appl. No.: 10/681,479

(22) Filed: Oct. 8, 2003

Related U.S. Application Data
(60) Provisional application No. 60/417,739, filed on Oct. 10, 2002.

(51) Int. Cl. C12Q 1/68; C12P 19/34

(52) U.S. Cl. 435/6; 435/91.2

(57) ABSTRACT

An mRNA is amplified by (a) binding a first primer to a target mRNA, the first primer comprising, in the 5′ to 3′ direction, a first known segment and an oligo T segment; (b) transcribing a cDNA from said target mRNA by elongation of said first primer with reverse transcriptase; and then (c) linking a second known segment to the 3′ terminus of said cDNA. In a preferred embodiment the step of transcribing a cDNA from said target mRNA is carried out so that at least one additional C residue is produced on the 3′ terminus of said cDNA, and the said step of linking a second known segment to the 3′ terminus of said cDNA is carried out by: (i) binding a second bridge primer to said cDNA, said second primer comprising, in the 5′ to 3′ direction, a second known segment and at least one G residue, said second primer having an inactivated G residue on the 3′ terminus thereof; and then (ii) further transcribing said cDNA from second bridge primer by elongation of said at least one additional C residue with reverse transcriptase so that a cDNA is produced having said first known segment on the 5′ terminus thereof and said second known segment on the 3′ terminus thereof. The method can be used to amplify a plurality of mRNAs together, even though a small sample of mRNAs is available such as obtained from a single cell or multiple cells obtained by laser capture microdissection from tissue or organs, and the product of the amplification then used for gene family analysis or microarray expression analysis.

### RELATED APPLICATIONS

This application claims the benefit of U.S. Provisional Application 60/417,739, filed Oct. 10, 2002, the disclosure of which is incorporated by reference herein in its entirety.

### STATEMENT OF GOVERNMENT SUPPORT

The present invention was made with U.S. Government support under grant number HL36059 from NHLBI and grant number HD17063 from NICHD. The Government has certain rights to this invention.

### FIELD OF THE INVENTION

The present invention concerns methods of amplifying mRNA, and particularly concerns methods of uniformly amplifying mRNA pools for subsequent gene expression profiling.

### BACKGROUND OF THE INVENTION

Gene profiling is the analysis of the dynamics of genome expression in biological systems. It will provide valuable insights as to the mechanisms that determine cell phenotype and function by establishing gene expression profiles on a genome globally for single or multiple cells from complex heterogeneous tissue. This kind of technology will undoubtedly be a cornerstone for functional genomics exploration. Through the information gained with gene profiling, it will be possible to quantitatively assess differences in gene expression in normal versus disease states. From this, novel drug targets can be identified in the potential treatment of these afflictions.

Two powerful technologies, laser capture microdissection (LCM) and DNA microarray gene profiling analysis, provide the means for such characterizations, but have not been exploited because the quality and quantity of RNA from captured cells is not optimal for microarray screening.

cDNA microarray technologies offer a highly parallel approach for profiling expressed gene sequences in disease-relevant tissues. However, standard hybridization and detection protocols are insufficient for milligram quantities of tissue, let alone a small population of cells such as those derived from needle biopsies and LCM. However, amplification systems utilizing T7 RNA polymerase can provide multiple cRNA or cDNA copies from mRNA transcripts, permitting microarray studies with reduced sample inputs (Pabon et al. (2001) Biotechniques 31: 874-879).

In The Scientist, pg. 37 (Jun. 24, 2002) it is stated that improved techniques reduce the amount of RNA required in a sample for subsequent microarray analysis from 20 μg total RNA for analysis to 1 μg total RNA (equivalent to about 100,000 cells) for analysis. Since it would be extremely useful to be able to utilize these techniques in situations where fewer than 100,000 cells are available for analysis, there is a need for new techniques to carry out gene profiling, microarray analysis, gene family analysis and the like with smaller cell populations and smaller RNA samples.

### SUMMARY OF THE INVENTION

A first aspect of the present invention is a method of amplifying an mRNA, comprising the steps of: (a) binding a first primer to a target mRNA, the first primer comprising, in the 5′ to 3′ direction, a first known segment and an oligo T segment; (b) transcribing a cDNA from the target mRNA by elongation of the first primer with reverse transcriptase; and then (c) linking a second known segment (e.g., a DNA) to the 3′ terminus of the cDNA. The target mRNA is preferably removed prior to subsequent amplification reactions as discussed further below.

In one embodiment of the foregoing, the step of transcribing a cDNA from the target mRNA is carried out so that at least one additional C residue is produced on the 3′ terminus of the cDNA; and the step of linking a second known segment to the 3′ terminus of the cDNA is carried out by: (i) binding a second bridge primer to the cDNA, the second primer comprising, in the 5′ to 3′ direction, a second known segment and at least one G residue, the second primer having an inactivated G residue on the 3′ terminus thereof; and then (ii) further transcribing the cDNA from second bridge primer by elongation of the at least one additional C residue with reverse transcriptase so that a cDNA is produced having the first known segment on the 5′ terminus thereof and the second known segment on the 3′ terminus thereof. The first and second primers, particularly the second primer, are preferably removed prior to subsequent amplification steps.

In another embodiment of the foregoing, the step of transcribing a cDNA from the target mRNA is followed by the step of adding at least one additional predetermined residue to the 3′ terminus of the cDNA with a terminal deoxynucleotidyl transferase; and the step of linking a second known segment to the 3′ terminus of the cDNA is carried out by: (i) binding a second bridge primer to the cDNA, the second primer comprising, in the 5′ to 3′ direction, a second known segment and at least one corresponding residue, which corresponding residue binds to the at least one additional predetermined residue by Watson-Crick pairing, the second primer having an inactivated predetermined residue on the 3′ terminus thereof; and then (ii) further transcribing the cDNA from second bridge primer by elongation of the at least one additional predetermined residue with reverse transcriptase so that a cDNA is produced having the first known segment on the 5′ terminus thereof and the second known segment on the 3′ terminus thereof. For example, the at least one additional unmatched residue may be selected from the group consisting of A, T, C, G, and oligomers thereof, and the at least one corresponding residue may be selected from the group consisting of A, T, C, G, and oligomers thereof. mRNA and the primers are preferably removed as necessary prior to subsequent amplification.

In another embodiment of the foregoing, the step of linking a second known segment to the 3′ terminus of the cDNA is carried out by directly linking the second known segment to the 3′ terminus with RNA ligase.

The method preferably further comprises the step of amplifying the cDNA (e.g., by polymerase chain reaction, ligase chain reaction, rolling circle amplification, other suitable amplification technique, and serial combinations thereof), preferably with said first and second known segments, such as with a pair of amplification primers, one of which pair binds to the first known segment and the other of which pair binds to the second known segment, or in an embodiment optimized for rolling circle amplification, with a first known segment and a second known segment that comprise the same nucleic acid sequence in opposite orientation.

A further aspect of the present invention is a method of uniformly amplifying a plurality of different target mRNAs in a sample, the method comprising the steps of: (a) binding a first primer to a each of the target mRNA, the first primer comprising, in the 5′ to 3′ direction, a first known segment and an oligo T segment; (b) transcribing a cDNA from the each of the target mRNA by elongation of the first primer with reverse transcriptase; then (c) linking a second known segment to the 3′ terminus of each of the cDNAs; and then (d) uniformly amplifying each of the cDNAs with a pair of primers, one of which pair binds to the first known segment and the other of which pair binds to the second known segment. The particular embodiments and features described above can be utilized where a plurality of mRNAs (e.g., at least 500, 1,000, 5,000, or 10,000 or more, up to 30,000, 60,000 or more different and distinct mRNA species) are found in the sample. The method may be advantageously utilized when small amounts of mRNA are found in the starting sample: e.g., wherein the target mRNAs on which the method is performed consists of or consists essentially of mRNA extracted from not more than 1, 10, 100 or 1,000 cells, and/or wherein the total amount of target mRNAs on which the method is performed (including all distinct species mixed together) consists of or consists essentially of not more than 1, 10 or 100 nanograms of mRNA, or not more than 1, 10 or 100 picograms of mRNA. The method preferably includes the step of (e) determining the quantity of each of at least a portion of the different cDNAs (e.g., 2, 5 or 10 or more, such as for gene family analysis; or 200, 500, 1,000 or 2,000 or more, such as for microarray analysis) to thereby provide an indication of the amounts of the corresponding mRNAs present in the sample (particularly the amounts relative to one another).

A further aspect of the present invention is a primer pair useful for amplifying an mRNA as described above or amplifying a plurality of mRNAs as described above, comprising: (a) a first primer, the first primer comprising, in the 5′ to 3′ direction, a first known segment and an oligo T segment; and (b) a second bridge primer, the second primer comprising, in the 5′ to 3′ direction, a second known segment and a binding segment, the binding segment selected from the group consisting of A, oligo A, T, oligo T, C, oligo C, G, and oligo G, the second primer having an inactivated residue on the 3′ terminus thereof. The primer pair may be packaged together in a kit, the kit optionally including printed instructions or printed reference to instructions for carrying out the methods described above.

A further aspect of the present invention is the use of a primer or pair of primers as described above in a method as described above.

The present invention is explained in greater detail in the drawings herein and the specification set forth below.

### BRIEF DESCRIPTION OF THE DRAWINGS

FIG. 1 is a schematic illustration of one embodiment of an amplification process of the present invention.

FIG. 2. The effects of different fixation methods on RNA stability. Whole embryos at stage 14 were fixed or frozen as listed below. Each lane represents RT-PCR of RNAs isolated from a single embryo. Lane M, 100 bp ladder; lanes 1-6, random primer RT; lanes 7-13, oligo (dT) primer RT. Lanes 1 and 7, 4.0% paraformaldehyde; lanes 2 and 8, 3% glutaraldehyde; lanes 3 and 9, 70% methanol; lanes 4 and 10, 95% ethanol/acetic acid; lanes 5 and 11, methacarn; lanes 6 and 12, fresh frozen in OCT (Tissue-Tek®).

FIG. 3. The effects of different fixation methods on RNA stability. Each lane shows RT-PCR of GAPDH RNA isolated from a single stage 14 embryo fixed or frozen as listed below. Lane M, 100 kb ladder; lanes 1-6, random primer RT; lanes 7-13, oligo (dT) primer RT; lanes 1 and 7, 4.0% paraformaldehyde; lanes 2 and 8, 3% glutaraldehyde; lanes 3 and 9, 70% methanol; lanes 4 and 10, 95% ethanol/acetic acid; lanes 5 and 11, methacarn; lanes 6 and 12, fresh frozen in OCT.

FIG. 4. Frozen sections of the pharyngeal region of a mouse embryo at day 8.5 of gestation. In (A) the dark line outlines the ventral midline endoderm of the ventral pharynx. The dark line is the melted tissue that circumscribes the targeted cells after the laser microdissection but before the tissue is captured. (B) shows the captured ventral midline endoderm. (C) shows a collection of the captured tissues. Stained samples of captured cells indicated that each capture represented 15-20 cells.

FIG. 5A shows a pilot test to determine the number of cycles needed for long distance PCR amplification of the cDNA pools.

FIG. 5B shows 2 μl of reverse transcription product from the laser microdissected cells of the ventral and dorsal midline of the pharyngeal endoderm was used to perform long distance PCR at 25 and 30 cycles.

FIG. 6. Gene expression profiles of ventral and dorsal pharyngeal endoderm in E7.5 mouse embryo. Gray area shows the gene expression of both sides at small level. Red lines indicate a five fold increase in expression and blue lines indicate a five fold decrease in expression.

FIG. 7. Whole mount in situ hybridization confirmed a microarray data suggested a gene differential expressed between ventral and dorsal midline of pharyngeal endoderm.

### DETAILED DESCRIPTION OF THE PREFERRED EMBODIMENTS

Nucleotide sequences are presented herein by single strand only, in the 5′ to 3′ direction, from left to right. Nucleotides and amino acids are represented herein in the manner recommended by the IUPAC-IUB Biochemical Nomenclature Commission, or (for amino acids) by three letter code, in accordance with 37 C.F.R § 1.822 and established usage. See, e.g., PatentIn User Manual, 99-102 (November 1990) (U.S. Patent and Trademark Office).

The disclosures of all United States patent and patent application references cited herein are to be incorporated by reference herein as if fully set forth.

An “inactivated residue” such as an inactivated G residue or inactivated predetermined residue as used herein refers to a 3′ terminal nucleotide on a polynucleotide, which 3′ terminal nucleotide is incapable of further elongation such as in a RNA reverse transcription and an DNA amplification reaction. In general, such an inactivated residue will be modified so that the 3′ hydroxy group is eliminated, such as by replacement of dideoxyGTP or by replacement with the H, loweralkyl, sulfhydryl or other suitable blocking group.

By “uniformly amplifying” is meant that the size of individual nucleic acid species (mRNAs or their corresponding cDNAs) in a pool is not substantially changed, and that the ratio of individual nucleic acid species within the pool with respect to one another is not substantially altered, during the amplification process. In general, such uniform amplification may be carried out by maintaining the amplification reaction process in the logarithmic or linear phase thereof by limiting the number of cycles and/or time of the reaction process so that the reaction does not reach a plateau phase. Where multiple different amplification reactions are performed on the nucleic acid pool, each reaction will preferably be carried out in the logarithmic phase.

The terms “primer,” “probe,” “nucleic acid” and “oligonucleotide” are used interchangeably herein and generally refer to at least two nucleotides covalently linked together. A nucleic acid of the present invention will generally contain phosphodiester bonds, although in some cases, as outlined below, nucleic acid analogs are included that may have alternate backbones, comprising, for example, phosphoramide (e.g., Beaucage et al. Tetrahedron 49 (10): 1925 (1993); phosphorothioate (Mag et al. Nucleic Acids Res. 19: 1437 (1991); and U.S. Pat. No. 5,644,048), phosphorodithioate (Briu et al. J. Am. Chem. Soc. 111: 2321 (1989)), O-methylphophoroamidite linkages (see Eckstein. “Oligonucleotides and Analogues: A Practical Approach,” Oxford University Press), and peptide nucleic acid backbones and linkages (see Egholm. J. Am. Chem. Soc. 114: 1895 (1992); Meier et al. Angew. Chem. Int. Ed. Engl. 31: 1008 (1992); Nielsen. Nature 365: 566 (1993); Carlsson et al. Nature 380: 207 (1996)). Other analog nucleic acids include those with positive backbones (Denpcy et al. Proc. Natl. Acad. Sci. U.S.A. 92: 6097 (1995)); non-ionic backbones (U.S. Pat. Nos. 5,386,023; 5,637,684; 5,602,240; 5,216,141; and 4,469,863; Kiedrowshi et al. Angew. Chem. Intl. Ed. English 30: 423 (1991); Letsinger et al. J. Am. Chem. Soc. 110: 4470 (1988); Letsinger et al. Nucleoside & Nucleotide 13: 1597 (1994); Chapters 2 and 3, ASC Symposium Series 580, “Carbohydrate Modifications in Antisense Research,” Ed. Y. S. Sanghui and P. Dan Cook; Mesmaeker et al. Bioorganic & Medicinal Chem. Lett. 4: 395 (1994); Jeffs et al. J. Biomolecular NMR 34: 17 (1994); Tetrahedron Lett. 37: 743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research,” Ed. Y. S. Sanghui and P. Dan Cook. Nucleic acids containing one or more carbocyclic sugars are also included within the definition of nucleic acids (see Jenkins, et al. Chem. Soc. Rev. (1995) pp. 169-176). Several nucleic acid analogs are described in Rawls. C & E News Jun. 2, 1997, page 35. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

A “first known segment” and a “second known segment” as described herein may be any sequence of nucleotide residues in an oligonucleotide, but are generally from 10 or 15 nucleotides in length up to 30, 40 or 50 nucleotides in length, or more. Such segments may serve as binding partners for other nucleic acid primers (e.g., amplification primers), may encode promoters, may have the same sequence as each other but in opposite orientation as when used for rolling circle amplification, etc.

An “oligomer” or “oligo” as used herein generally refers to a polynucleotide comprising a repeating sequence of the same nucleotide base, such as oligo A, oligo T, oligo C, olig G. Such oligomers may be of any suitable length, such as from 2 or 3 nucleotides in length up to 30 or 40 nucleotides in length, or more.

“Amplification primers” as used herein refer to oligonucleotides, generally from 10 or 15 nucleotides in length up to 30, 40 or 50 nucleotides in length, or more, used as reagents to carry out an amplification reaction as discussed further below.

mRNA Samples. The mRNA used to carry out the present invention may be obtained from any suitable source, including plant, animal, and microbial cells (e.g., bacterial, protozoal, fungal, etc.). In one embodiment, the cells are collected by laser capture microdissection, enabling a small number of homologous cells to be collected (e.g., 1, 10, 100 or 1,000 cells). The mRNA can be extracted from the cells by any suitable technique that sufficiently preserves the structure and integrity thereof for subsequent amplification and analysis. In preferred embodiments such as used for gene profiling or gene family analysis, the total mRNA, representing the gene pool of the cell, is extracted from the cell or cells for subsequent analysis.

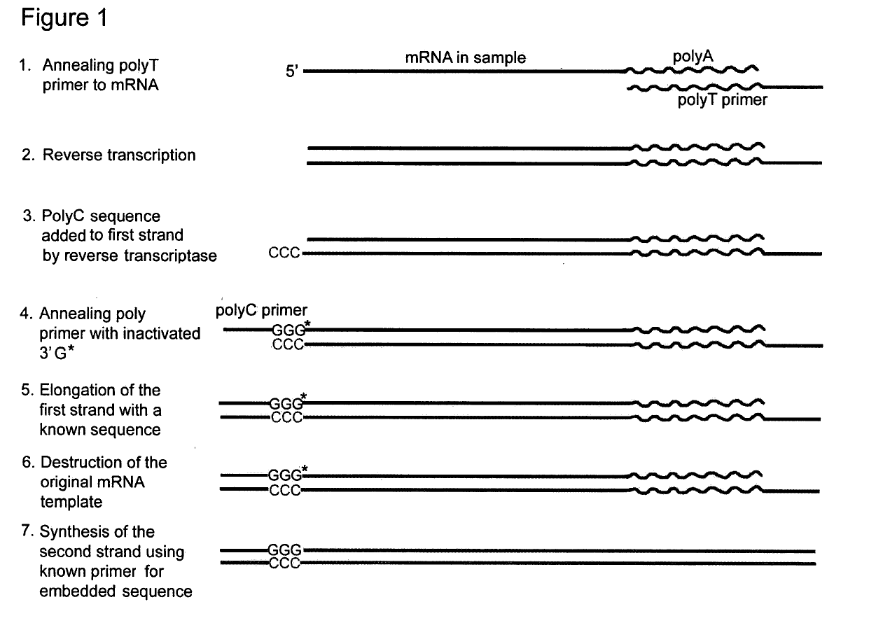

An example of one embodiment of the present invention is schematically illustrated in FIG. 1. As shown in part 1 of FIG. 1, a first primer is bound or annealed to a target or template mRNA. The first primer comprises, in the 3′ to 5′ direction, an oligo T segment and a first known segment (e.g., a primer binding region, or a promoter segment such as a T7 promoter, depending upon subsequent amplification reactions employed).

As shown in part 2, A cDNA is then reverse transcribed by elongation of the first primer, utilizing the target mRNA as a template, by any suitable technique (typically with a reverse transcriptase).

Part 3 of FIG. 1 illustrates that, as a consequence of reverse transcription, a oligo C segment is added to the 3′ terminus of the cDNA. As shown in part 4, a second “bridge” primer is then bound or annealed to the target mRNA, the bridge primer comprising, in the 3′ to 5′ direction, an oligo G segment and a second known segment (e.g., an SP6 segment). Note that the bridge primer has a 3′ G residue that is blocked or inactivated so as to prevent cDNA synthesis in the 3′ direction therefrom.

As shown in parts 4-5, the addition of the bridge primer allows elongation of the cDNA to continue by reverse transcription as in part 2 above, but now adding the complement to the second known sequence to the 3′ end of the cDNA. At this point, note that the cDNA now has the first known segment from the first primer at the 5′ terminus thereof, and the complement of the second known segment (from the second or “bridge” primer) at the 3′ terminus thereof.

As shown in parts 6-7, the template mRNA is removed or destroyed and a complementary strand synthesized utilizing the cDNA as the template, by any suitable technique.

Inactivated or chain-terminating nucleotides and labels. A Nucleotide 5′-triphosphates are substrates of polymerase enzymes and are incorporated into DNA by chain elongation or extension by internucleotide phosphodiester bond formation between the 3′ hydroxyl terminus of the extending chain and the 5′ hydroxyl of the nucleotide. Further extension by incorporation of more nucleotide 5′-triphosphates requires a new 3′ hydroxyl terminus. During chain extension, typically a mixture of nucleotide 5′-triphosphates are present, e.g., dATP, dGTP, dCTP and dTTP. Labeled nucleotides may also be present, for detection, isolation, or immobilization of the newly synthesized DNA. Nucleotides which terminate extendability (“terminators” or “terminating nucleotides”) by blocking the incorporation of additional nucleotides into an elongating chain may also be present in the mixture. Exemplary terminators include, but are not limited to, 2′,3′-dideoxynucleotides (ddNTP), 2,3′-dideoxy-dehydronucleotides, nucleotide analogs, such as a fructose based nucleotide analog, or a chemically modified purine or pyrimidine that retains the ability to specifically base-pair with naturally occurring nucleotides may be used to block DNA polymerization. A variety of 3′-substituted nucleotides (Antrazhev (1987) Bioorg. Khim. 13: 1045-52; Chidgeavadze et al. (1986) Biochim. Biophys. Acta 868: 145-52; Chidzhacadze et al. (1989) Mol. Biol. (Mosk.) 23: 173-42), such as azido-(Mitsuya et al. (1986) Proc. Natl. Acad. Sci. USA 83: 1191), mercapto-(Yuzhakov et al. (1992) FEBS Letters 306: 185-88), amino-(Herrein et al. (1994) Helvetica Chimica Acta 77: 586-96), and fluoro-(Chidgeavadze et al. (1985) FEBS Letters 183: 975-8) substituted nucleotides, which have been reported to terminate DNA synthesis, may be used in the present invention.

Labeled terminators are particularly useful in the present invention. Labels provide a signal for detection of labeled DNA products by fluorescence, chemiluminescence, and electrochemical luminescence (Kricka (1992) In: Nonisotopic DNA Probe Techniques, Academic Press, San Diego, pp. 3-28). Chemically linking labels to nucleotides is well-known in the art (e.g., U.S. Pat. Nos. 4,811,218 and 4,855,225). Exemplary chemiluminescent labels are 1,2-dioxetane compounds (U.S. Pat. No. 4,931,223; Bronstein et al. (1994) Anal. Biochem. 219: 169-81). Fluorescent dyes useful for labeling nucleotide 5′-triphosphates include fluoresceins (Menchen et al. (1993) U.S. Pat. No. 5,188,934), rhodamines (U.S. Pat. No. 5,366,860), cyanines (WO 97/45539), and metal porphyrin complexes (WO 88/04777).

Fluorescein dyes are well-known in the art and include, but are not limited to, 6-carboxyfluorescein (6-FAM); 2′,4′,1,4,-tetrachlorofluorescein (TET); 2′,4′,5′,7′,1,4-hexachlorofluorescein (HEX); 2′,7′-dimethoxy-4′,5′-dichloro-6-carboxyrhodamine (JOE); 2′-chloro-5′-fluoro-7′,8′-fused phenyl-1,4-dichloro-6-carboxyfluoresccin (NED); and 2′-chloro-7′-phenyl-1,4-dichloro-6-carboxyfluorescein (VIC), CY3™, CYS™, CY3.5™, CY5.5™ and the like The 5-carboxyl, and other regio-isomers, may also have useful detection properties.

Another preferred class of labels include fluorescence quenchers. The emission spectra of a quencher overlaps with a proximal intramolecular or intermolecular fluorescent dye such that the fluorescence of the fluorescent dye is substantially diminished, or quenched, by the phenomena of fluorescence resonance energy transfer “FRET” (Clegg (1992) Meth. Enzymol. 211: 353-388). Particularly preferred quenchers include, but are not limited to, rhodamine fluorescent dyes (e.g., tetramethyl-6-carboxyrhodamine (TAMRA), tetrapropano-6-carboxyrhodamine (ROX)) and cyanine dyes (e.g., nitrothiazole blue (NTB), anthraquinone, malachite green, nitrothiazole, and nitroimidazole compounds).

Amplification. Amplification steps utilized in carrying out the present invention may be implemented by any suitable means. See generally D. Kwoh and T. Kwoh, Am. Biotechnol. Lab. 8, 14-25 (1990). Examples of suitable amplification techniques include, but are not limited to, polymerase chain reaction, fluorescent oligonucleotide dendrimeric signal amplification, ligase chain reaction, strand displacement amplification (see generally G. Walker et al. Proc. Natl. Acad. Sci. USA 89, 392-396 (1992); G. Walker et al. Nucleic Acids Res. 20, 1691-1696 (1992)), transcription-based amplification (see D. Kwoh et al. Proc. Natl. Acad. Sci. USA 86, 1173-1177 (1989)), self-sustained sequence replication (or “3SR”) (see J. Guatelli et al. Proc. Natl. Acad. Sci. U.S.A. 87, 1874-1878 (1990)), the Q.beta. replicase system (see P. Lizardi et al. BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). Polymerase chain reaction, alone or in combination with other techniques, is currently preferred.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or microarray analysis as described further below.

Strand displacement amplification may be carried out in accordance with known techniques. See, e.g., U.S. Pat. Nos. 5,712,124; 5,744,311; and 5,648,211. In one embodiment, strand displacement amplification may be carried out on a target nucleic acid sequence by a method comprising: (a) providing a single stranded nucleic acid fragment containing the target nucleic acid sequence, the fragment having a 5′ end and a 3′ end (e.g., a cDNA as described herein); (b) binding an oligonucleotide primer to the 3′ end of the fragment such that the primer forms a 5′ single stranded overhang, the primer comprising a 3′ end complementary to the 3′ end of the fragment and a 5′ end comprising a recognition sequence for a restriction endonuclease which does not cut the target nucleic acid sequence; (c) extending the primer on the fragment in the presence of (i) a DNA polymerase lacking 5′-3′ exonuclease activity, (ii) deoxynucleoside triphosphates, (iii) at least one substituted deoxynucleoside triphosphate, and (iv) a restriction endonuclease which nicks the recognition sequence when the recognition sequence is double stranded and hemimodified by incorporation of the substituted deoxynucleoside triphosphate, thereby producing a double stranded first reaction product comprising the primer, a first newly synthesized strand and a hemimodified restriction endonuclease recognition sequence; (d) nicking the double stranded hemimodified restriction endonuclease recognition sequence with the restriction endonuclease; (e) extending from the nick using the polymerase, thereby displacing the first newly synthesized strand from the first reaction product and generating a second newly synthesized strand, and (f) repeating the nicking, extending and displacing steps such that the target sequence is amplified.

Rolling circle amplification may be carried out in accordance with known techniques, including but not limited to those described in U.S. Pat. Nos. 6,344,329; 6,287,824; 6,235,502; 6,210,884; and 5,854,033 (Lizardi). In one embodiment, rolling circle amplification is carried out by (a) mixing a rolling circle replication primer with one or more amplification target circles (ATC), to produce a primer-ATC mixture, and incubating the primer-ATC mixture under conditions that promote hybridization between the amplification target circles and the rolling circle replication primer in the primer-ATC mixture, wherein the amplification target circles each comprise a single-stranded, circular DNA molecule comprising a primer complement portion, and wherein the primer complement portion is complementary to the rolling circle replication primer, wherein at least one of the amplification target circles is tethered to a specific binding molecule so that the amplification target circle can rotate freely (e.g., prepared by including first and second known segments on the cDNA as described herein, which first and second known segments comprise the same sequence in opposite orientation), (b) mixing DNA polymerase with the primer-ATC mixture, to produce a polymerase-ATC mixture, and incubating the polymerase-ATC mixture under conditions that promote replication of the amplification target circles, wherein replication of the amplification target circles results in the formation of tandem sequence DNA, and, simultaneous with, or following, step (b), (c) mixing RNA polymerase with the polymerase-ATC mixture, and incubating the polymerase-ATC mixture under conditions that promote transcription of the tandem sequence DNA, wherein transcription of the tandem sequence DNA results in the formation of transcript RNA.

Fluorescent oligonucleotide dendrimeric signal amplification or 3DNA amplification may be carried out in accordance with known techniques as described in, for example, Nilsen, T. W., Grazel, J., Prensky, W., “Dendritic Nucleic Acid Structures,” J. Theoretical Biology, 187: 273-284 (1997); Capaldi, S., Getts, R. C., and Jayasena, S. D., “A Signal Amplification Through Nucleotide Extension and Excision on a Dendritic DNA Platform,” Nucl. Acids Res., 28 (7): 21e (2000); Wang, J., Jiang, M., Nilsen, T. W., and Getts, R., “Dendritic Nucleic Acid Probes for DNA Biosensors,” J. Am. Chem. Soc., 120: 8281-8282 (1998); Wang, J., Rivas, G., Fernandes, J., Jiang, M., Lopez Paz, J. L., Waymire, R., Nilsen, T. W., and Getts, R., “Adsorption and Detection of DNA Dendrimers at Carbon Electrodes,” Electroanalysis, 10 (8): 553-556 (1998); R. Stears et al. Physiol. Genomics 3, 93-99 (2000); and R. Getts, US Patent Application 20020051981 (May 2, 2002). Kits and materials for carrying out such methods are available from Genisphere Inc. 2801 Sterling Drive, Hatfield, Pa. 19440 USA (Telephone No. 215-996-3002). In general, construction of a 3DNA dendrimer begins with a single initiator monomer. To this, a first layer of monomers is attached by annealing the “arms” of the first layer monomers to the “arms” of the initiator monomer. The result is a one-layer 3DNA dendrimer with a plurality (e.g., 12) single-strand “arms” available on its surface. This structure is then chemically crosslinked to prevent dissociation. Next, a second layer of monomers is attached to the first layer using the same interaction between the single-stranded “arms” of each component. In this two-layer 3DNA dendrimer the number of free single-stranded “arms” increases (e.g., to 36). A third layer of monomers is added in an analogous fashion, creating a total of, for example, 108 free single-stranded arms. Finally, in a four-layer dendrimer, addition of the last set of monomers leaves, for example, up to about 324 single-stranded “arms” on the surface of the molecule. The “arms” on the surface of the four-layer 3DNA dendrimer are used to attach the dendrimer's two key functionalities. One function of the arms is to enable attachment of label. The other is to make the dendrimer specific to a particular application or experiment.

A first amplification step may be followed by one or more additional amplification steps, such as in vitro transcription of DNA or mRNA from a promoter such as a T7 promoter included within the first known segment.

Microarray formation. The nucleic acid fragments generated in accordance with the teachings of the present invention are intended to be made into an array wherein each individual fragment is deposited in a defined space and location on a solid or semi-solid support. As used herein, an array is an orderly arrangement of nucleic acid fragments, as in a matrix of rows and columns or spatially addressable or separable arrangement such as with coated beads. With an automated delivery system, such as a Hamilton robot or ink-jet printing method, one can form a very complex array of nucleic acid fragments on a solid support, for example an epoxysilane, mercaptosilane or disulfidesilane-coated solid support. Such methods can deliver nano to pico-liter size droplets with sub-millimeter spacing. Arrays typically have a surface density of at least 10 distinct and separated nucleic acid fragments per square centimeter and up to 10³ or more distinct and separated spotted nucleic acid fragments per square centimeter. Such arrays can be assembled through the use of a robotic liquid dispenser (such as an ink-jet printing device controlled by a piezoelectric droplet generator) such that each nucleic acid molecule occupies a spot of more than about 10 microns or more than 25 microns in diameter and each nucleic acid spot is spaced no closer, center to center, than the average spot diameter. Methods and apparatuses for dispensing small amount of fluids using such ink-jet printing techniques and piezoelectric ink-jet depositions are well-known to one of skill in the art (see e.g., U.S. Pat. Nos. 4,812,856; 5,384,261; 5,405,783; 5,424,186; 5,599,695; 5,807,522; 5,800,992; 6,004,755; and 6,087,1020).

The array may further be constructed using the method of Fodor, et al. (U.S. Pat. No. 5,445,934). Fodor, et al. provides a method for constructing an array onto a solid surface wherein the surface is covered with a photo-removable group. Selected regions of the substrate surface are exposed to light to as to activate the selected regions. A monomer, which also contains a photo-removable group, is provided to the substrate surface to bind to the selected area. The process is repeated to create an array.

The array may further be created by means of a “gene pen.” A “gene pen,” as used herein, refers to a mechanical apparatus comprising a reservoir for a reagent solution connected to a printing tip. The printing tip further comprises a means for mechanically controlling the solution flow. Under one embodiment, a multiplicity of “gene pens” or printing tips may be tightly clustered together into an array, with each tip connected to a separate reagent reservoir. Under another embodiment, discrete “gene pens” may be contained in an indexing turntable and printed individually. Typically, the solid surface is pretreated to enable covalent or non-covalent attachment of the reagents to the solid surface. Preferably, the printing tip is a porous pad.

Alternatively, the array may be created with a manual delivery system, such as a pipetman. Because these arrays are created with a manual delivery system, these arrays will not be as complex as those created with an automated delivery system. Arrays created with a manual delivery system will typically be spaced, center to center, 2 mm apart. Arrays created with a manual delivery system will be created in a 96-well or 384-well plate. Therefore, depending on the delivery system employed, one may create arrays spaced, center to center, with spacing ranging from 50 μm to 2 mm spacing.

Detection methods. The present invention further provides methods of detecting and quantifying the nucleic acid sequence fragments of the invention which are deposited on an array. Detection methods which may be used are dependent on the terminator label used and include, but are not limited to, enzyme-based detection, autoradiography, mass spectrometry, electrical methods, detection of absorbance or luminescence (including chemiluminescence or electroluminescence). Preferably, fluorescent labels are detected, for example, by imaging with a charge-coupled device (CCD) or fluorescence microscopy (e.g., scanning or confocal fluorescence microscopy), or by coupling a scanning system with a CCD array or photomultiplier tube, or by using array-based technology for detection (e.g., surface potential of each 10-micron part of a test region may be detected or surface plasmon resonance may be used if resolution can be made high enough.)

Alternatively, an array which contains nucleic acid sequences labeled with, for example, one of a pair of energy transfer probes, such as fluorescein and rhodamine, can be detected by energy transfer to, or modulation by, the label on a linker, target or reporter. Among the host of fluorescence-based detection systems are fluorescence intensity, fluorescence polarization (FP), time-resolved fluorescence, fluorescence resonance energy transfer and homogeneous time-released fluorescence (HTRF).

Analysis of the array output can be accomplished by pattern recognition followed by quantification of the intensity of the labels. Pattern recognition for the analysis of an array is similar to repeating bar-code patterns wherein the appropriate spot or line for each specific labeled target is found by its position relative to the other spots or lines. Bar-code recognition devices and computer software for the analysis of one or two-dimensional arrays are routinely generated and/or commercially available (e.g., see U.S. Pat. No. 5,545,531).

Methods of making and using the arrays of this invention, including preparing surfaces or regions, synthesizing or purifying and attaching or assembling substances such as those of the nucleic acid fragments described herein, and detecting and analyzing labeled or substances as described herein, are well-known and conventional technology. In addition to methods disclosed in the references cited above, see, e.g., patents assigned to Affymax, Affymetrix, Nanogen, Protogene, Spectragen, Millipore and Beckman (from whom products useful for the invention are available); standard textbooks of molecular biology and protein science, including those cited above; and U.S. Pat. No. 5,063,081; Southern (1996) Current Opinion in Biotechnology 7: 85-88; Chee, et al (1996) Science 274: 610-614; and Fodor, et al (1993) Nature 364: 555-556.

The present invention is explained in greater detail in the following non-limiting examples.

### EXAMPLE 1

### RNA Amplification Protocol

Primers. Primers used are as follows:

3′ primer:

(SEQ ID NO:1)

GAGTGAATTGTAATACGACTCACTATAGGGAAGCGG-d (T)₂₂.
5′ primer (bridge):

(SEQ ID NO:2)

5′-GATTTAGGTGACACTATAGAATAGGG-CH₃:
or

(SEQ ID NO:3)

5′-GATTTAGGTGACACTATAGAATAGGddG

Reverse transcription. Reverse transcription of the mRNA is carried out in accordance with known techniques by first annealing the reverse primer with the RNA template(s) under the following conditions:

| | | |
|---|---|---|
| Total RNA: | 1-9 | μl |
| T7/d(T) primer: | 1 | μl (1 pmol) |
| Add water to: | 10.5 | μl |

The mixture is reacted at 75° C. for 5 minutes, then put on ice for 2 minutes.

Transcription mix. The transcription mix is prepared in accordance with known techniques under the following conditions:

| | | |
|---|---|---|
| 5X RT buffer: | 4 | μl |
| 0.1 M DTT: | 2 | μl |
| 10 mM dNTPs | 2 | μl |
| RNasin (40u/μl) | 0.5 | μl |

The two parts (19.0 μl) are then mixed and put in 37° C. for 5 minutes, after which 1 μl (200 U) of RNA reverse transcriptase is added.

The reaction tube is then held at 42° C. for 15 minutes, after which 1 μl (10 pmol) of the 5′ bridge primer is added. Incubation is then continued at 37° C. for one or one and on-half hours.

RNA complementary to the cDNA, is then removed by adding add 1 μl (2 units) of Escherichia coli RNase H and incubate at 37° C. for 20 minutes. Primers and salts are then removed by Microcon YM-30 and the final volume is adjusted to to 20 μl.

Long region cDNA PCR. The polymerase chain reaction is carried out in accordance with standard techniques under the following reaction conditions.

| | | |
|---|---|---|
| First-Strand cDNA | 2 | μl |
| Deionized H₂O | 80 | μl |
| 10X PCR buffer | 10 | μl |
| 50X dNTP Mix | 2 | μl |
| 5' PCR Primer | 2 | μl |
| 3' PCR Primer | 2 | μl |
| 50X Advantage 2 Polymerase Mix | 2 | μl |
| Total volume | 100 | μl |

The tube is gently flicked to mix the contents and centrifuged briefly to collect the contents at the bottom of the tube. The tube is then placed in a preheated (95° C.) thermal cycler, and PCR amplification performed by following program:

94° C. 1 minute, then 16 cycles of 95° C. 8 seconds, 68° C. 6 minutes. The number of cycles is dependent on the initial amount of RNA in the sample and requires a pre-test for different situations such as a quantitative real time PCR of the house keeper genes. In one set of preferred embodiments the number of cycles is as follows:

| Total RNA (ng) | Number of Cycles |
|---|---|
| 250-500 | 12-14 |
| 50-250 | 14-16 |
| 10-50 | 16-18 |
| 1-10 | 18-20 |
| 0.01-1 | 20-22 |

After PCR, a 10 μl sample of the PCR product is analyzed on a 1.0% agarose/EtBr gel, alongside a 100 bp to 10 kb DNA size marker.

The cDNA resulting from the PCR is extracted once with phenol/chloroform, purified and concentrated with a Microcon™ YM-100 spin column (Millipore), and the cDNA volume adjusted to 15 μl.

Biotin labeling of RNA probes. To generate the biotinylated RNA probes for the DNA microarray, 0.5-1.0 μg cDNA is required to perform the in vitro transcription. A preferred kit for this task is the Enzo BioArray High Yield RNA Transcript Labeling System (Enzo, N.Y., USA). Microarray analysis follows the manufacturer's protocol. We use the Affymatrix oligonucleotides array and their work station.

### EXAMPLE 2

### Laser Microdissection Captured Cell Gene Expression Profile Analysis: Identification of Three Molecules involved in Ventral Axis Determination and Association with Three Human Genetic Syndromes

This example is provided to demonstrate the power of the instant invention in amplifying and profiling mRNA from small samples. This example demonstrates the application of the present invention to investigate a developmental event in which the ventral midline of pharyngeal endoderm serves as a ventral axis to coordinate the normal development of the head and heart in the chick embryo. To utilize the benefit of DNA microarray analysis we switched to mouse embryo, microdissected 10-15 cells from the ventral midline and the dorsal midline, and established gene expression profiles for each. The differential expression profile was then generated by using computerized subtraction of these two gene profiles. In the top ten listed targets, three genes were found to be involved in three different human syndromes, all of which shared common symptoms involving head and heart midline defects. These three genes and syndromes were: The Lim Kinase gene for William-Beuren syndrome, the Pegl/MEST gene for the Silver-Russell syndrome, and the SHFM gene for Split hand/foot malformation.

Rationale. The secondary heart field is most likely set aside at the time of gastrulation as a cardiogenic field. We know that the chick secondary heart field expresses both Nk×2.5 and Gata-4 which are also expressed by the primary heart fields. Thus, it is assumed that many of the same messages in the secondary heart field will be similar to those expressed in the primary heart fields prior to differentiation. But because differentiation of this region is delayed, it should also express genes that would delay differentiation into myocardium. Furthermore, identification of the precise time that myocardial genes are expressed will allow correlation of myocardial induction and differentiation with the availability of inductive factors in the pharynx. Previous studies have shown molecular differences in outflow versus ventricular myocardium (Ruzicka and Schwartz, 1988). It is not known if the myocardium from the secondary heart field undergoes a program of molecular differentiation distinct from that of the primary myocardium or if it is merely on a different time schedule. If true differences can be found in myocardium derived from primary versus secondary heart fields, these could be of importance in understanding other aspects of outflow development.

Protocol: We will use three time points for comparison: each field prior to myocardial differentiation, during the process of differentiation, and at a point after expression of MF20 antigen. Since it will be necessary to use mouse embryos for screening microarrays, we will use previously published information for the primary heart fields to determine the best collection times. For the secondary heart field collections, we will use the data obtained in aim 1 of this proposal. The expression profile of myocardium from the caudal, proximal and distal outflow tract will be compared with myocardium from the presumptive right and left ventricles at stages 14, 22 and 28. By comparing myocardial expression over such a long developmental window, we should be able to determine whether the expression profile of outflow myocardium is merely different because of the delay in differentiation or represents a real difference in myocardium derived from primary versus secondary heart fields. We will use laser capture microdissection of cells from outflow versus ventricular myocardium, and compare the gene expression patterns using microarray screening. Mouse embryos at the appropriate stages of development are collected, rapidly frozen and sectioned. The frozen sections are placed on slides and melted only enough to attach them to 1″×3″ precleaned histological slides. The primary and secondary heart fields, outflow and ventricular myocardium are identified. The appropriate tissue is microdissected, captured, and the mRNA extracted and reverse transcribed. The microarray protocol is detailed in the methods section.

Laser-capture microdissection. Under RNase-free conditions, appropriately staged mouse embryos will be embedded with OCT medium in cryomold and frozen in dry ice cooled 2-metnylbutane (−60° C.). Each embryo is sectioned at 7-10 μm in a cryostat maintained at −25° C. The sections are mounted on RNase-free microscope slides and immediately frozen on a block of dry ice. The sections are stored at −80° C., if LCM is to be performed that day. Just prior to the LCM procedure, the slides are fixed twice in absolute ethanol for one minute. Rehydration is followed by steps of 95%, 70% and 50% ethanol in Rnase-free deionized water. One wash is done with purified water and then the slides are stained with Mayer's hematoxylin (1-2 min.), washed once in water (5 seconds), placed in blueing reagent (30-60 sec), 70% ethanol for 20 seconds, 95% ethanol for 20 seconds, Eosin Y (1-2 min.), then 95% ethanol wash (x2), 100% ethanol wash, xylene wash for 1 min. Finally, the slides are air dried for at least 2 min to allow the xylene to evaporate completely.

Laser Transfer is performed using the PALM Robot-Microbeam system (Zeiss/P.A.L.M. Microlaser Technologies). Following the manufacture's protocols, the target tissue or cell is dissected out from the embryo sections. The tissue is captured on CapSure HS high sensitivity LCM transfer film manufactured by Arcturus. Total RNA is extracted from the captured samples using the Rnaeasy kit (Qiagene). Using 50 ng total RNA, RNA amplification is carried out as described in Example 1 above.

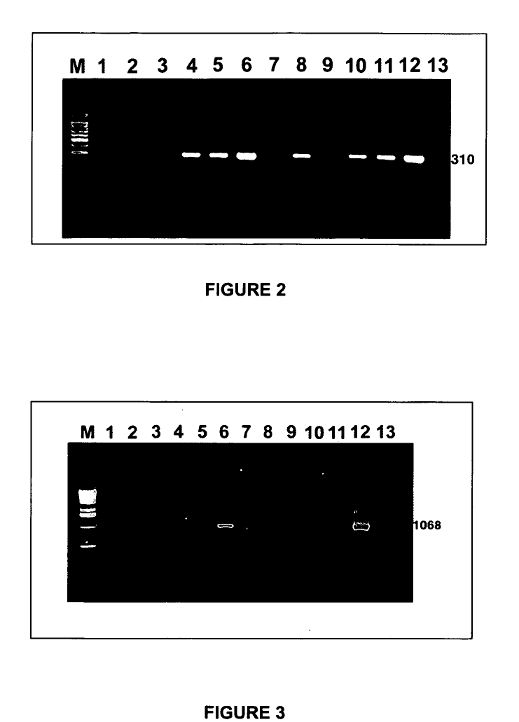

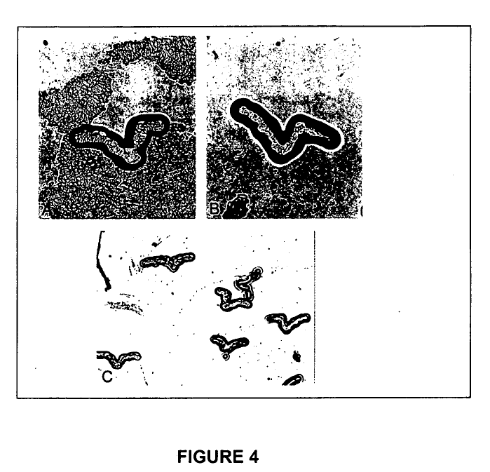

Optimization of the conditions for laser capture microdissection of multiple cells for gene profiling. Five different tissue fixation methods had been compared for laser capture microdissection based on the RNA quantity (FIG. 2) and quality (FIG. 3). However, the longest and most abundant RNAs were obtained from fresh frozen tissue.

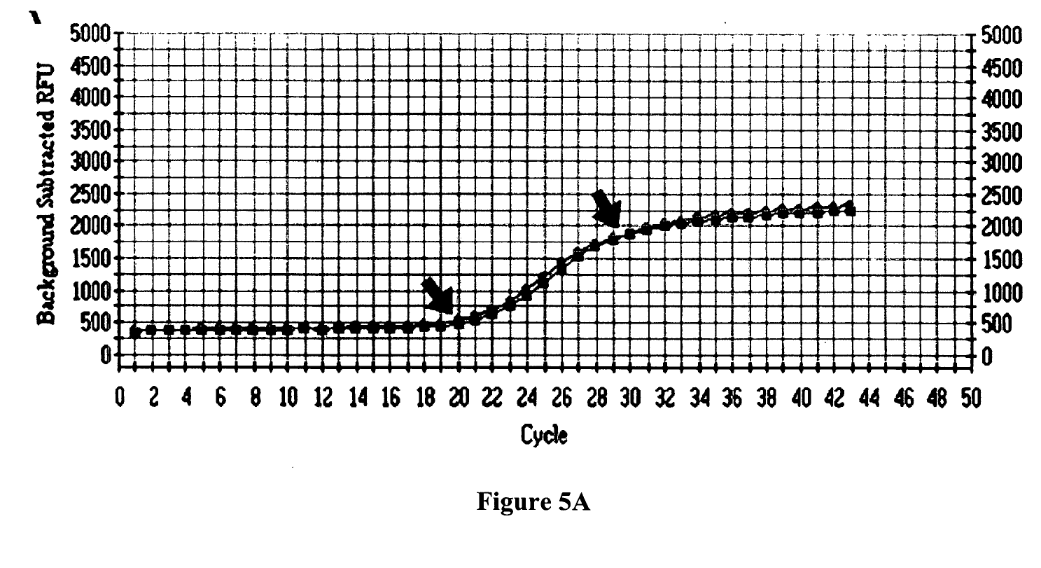

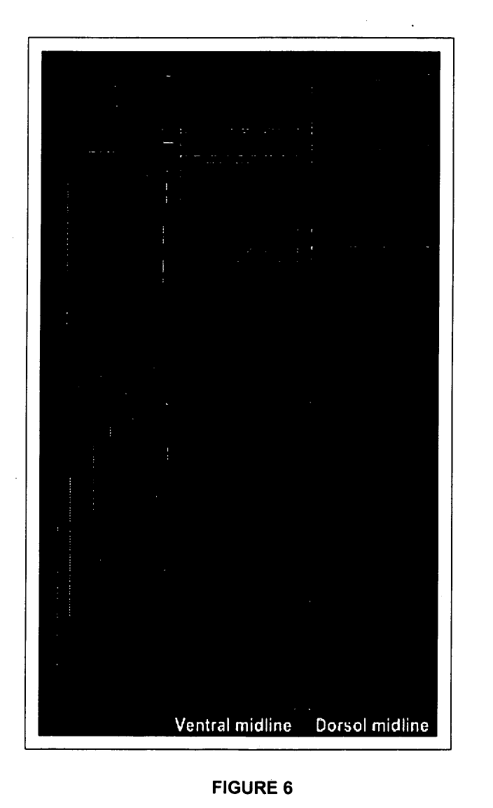

Gene expression profiles and the analysis of our microarray data. Our interest in ventral pharyngeal development has led us to ask whether the ventral pharyngeal endoderm differentially expresses messages as compared to the dorsal pharyngeal endoderm. This inquiry lends itself to laser capture microdissection and microarray screening. Laser capture microdissection was performed on fresh frozen sections from day 8.5 mouse embryos. The regions targeted for comparison were dorsal midline versus ventral midline foregut endoderm. FIG. 4 shows the steps in the capture of the ventral endoderm. RNA was extracted and cDNA prepared by the method described in the methods section of this application. The gene profiles of the GeneSpring data analysis platform provides systems operation, instrument control and data analysis for the entire genechip. It automatically acquires and processes hybridization data, analyzes algorithms and then allows review, comparison, graphing, filtration, analysis and reporting in different modes. Every sample has its own expression profile database. All the comparison and subtraction analyses are performed by the software. A two-fold differential expression of genes is listed and sorted gene tree based on gene function or family showed as FIG. 6 of the profiles of ventral and dorsal pharyngeal endoderm.

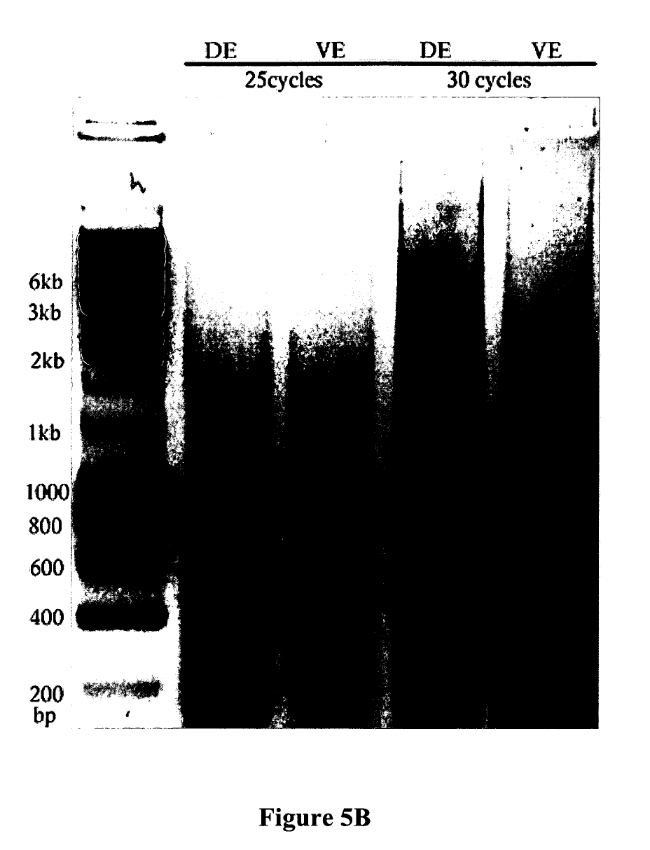

FIG. 5A shows a pilot test to determine the number of cycles needed for long distance PCR amplification of the cDNA pools. The highly abundant housekeeping gene GAPDH A was chosen to perform quantitative real time PCR on cDNA generated from the microdissected cells of the dorsal and ventral midline of the pharyngeal endoderm. The linear range or log phase of the amplification is between cycles 20-28 as indicated by the arrows. FIG. 5B shows 2 μl of reverse transcription product from laser microdissected cells of the ventral and dorsal midline of the pharyngeal endoderm, used to perform long distance PCR at 25 and 30 cycles. The amplified pools of cDNA are uniformly distributed in a smear between 100 bp to 6000 bp as shown in the agarose gel. These data indicated that 30 cycles of PCR caused over-amplification of the cDNA pool. The products from 25 cycles of amplification were used for continuing microarray analysis.

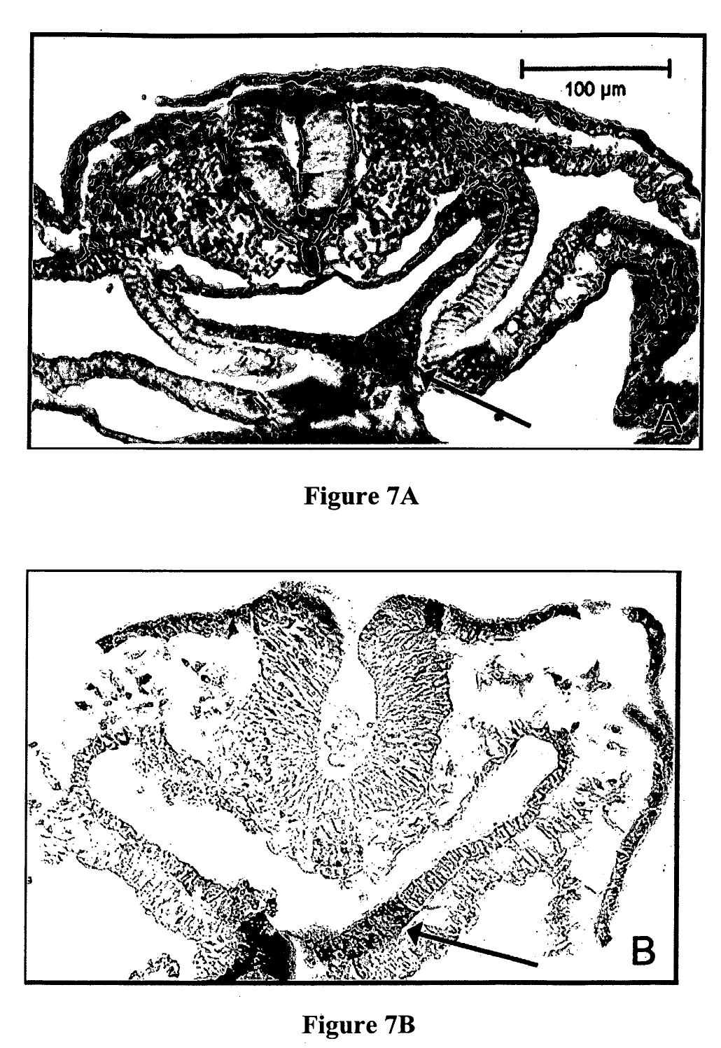

FIG. 7 shows that whole mount in situ hybridization confirmed microarray data suggesting differential gene expression between the ventral and dorsal midline of pharyngeal endoderm. FIG. 7A: DiI/CRSE injection in chick embryo stage 5 traced cells from the prechordal plate and generated the ventral midline of the pharyngeal endoderm at stage 12, but not the dorsal midline or other regions of the pharyngeal endoderm. FIG. 7B: After DNA microarray analysis and comparison of the gene expression profiles, there are many genes differentially expressed between the dorsal and ventral midline of the pharyngeal endoderm. This figure shows one example which was indicated by the microarray data, and confirmed it by in situ hybridization in chick embryo at stage 12, in which this gene was highly and specifically expressed in the ventral midline but not the dorsal midline as arrow pointed.

A list of some of the differentially expressed messages in the ventral midline pharyngeal endoderm is shown in Table 1. This is also the first demonstration that ventral versus dorsal foregut endoderm show differential gene expression. Interestingly, three top listed target genes are involved three different individual human syndromes, but shearing some common symptoms appearing head and heart midline defects.

TABLE 1
Messages showing the largest differences in microarray screen
comparing ventral versus dorsal foregut endoderm.

| Gene | Fold Change | Function or disease association |
|---|---|---|
| Lim Kinase | +52.4 | William-Beuren syndrome |
| Peg1/MEST | +19.5 | Silver-Russell syndrome |
| SHFM | +11.1 | Split hand/foot malformation |
| TPT1(P21) | +23.1 | Rho family Kinase activator |
| Ras-GTPase | +32.2 | LIMK pathway for morphological |
| AP3 | +25.2 | Membrane traffic |

The foregoing is illustrative of the present invention, and is not intended to be limiting thereof. The invention is defined by the following claims, with equivalents of the claims to be included therein.

1. A method of amplifying an mRNA, comprising the steps of:
(a) binding a first primer to a target mRNA, the first primer comprising, in the 5′ to 3′ direction, a first known segment and an oligo T segment;
(b) transcribing a cDNA from said target mRNA by elongation of said first primer with reverse transcriptase; and then
(c) linking a second known segment to the 3′ terminus of said cDNA.

2. The method of claim 1, wherein:
said step of transcribing a cDNA from said target mRNA is carried out so that at least one additional C residue is produced on the 3′ terminus of said cDNA; and
said step of linking a second known segment to the 3′ terminus of said cDNA is carried out by:
(i) binding a second bridge primer to said cDNA, said second primer comprising, in the 5′ to 3′ direction, a second known segment and at least one G residue, said second primer having an inactivated G residue on the 3′ terminus thereof; and then
(ii) further transcribing said cDNA from second bridge primer by elongation of said at least one additional C residue with reverse transcriptase so that a cDNA is produced having said first known segment on the 5′ terminus thereof and said second known segment on the 3′ terminus thereof.

3. The method of claim 1, wherein:
said step of transcribing a cDNA from said target mRNA is followed by the step of adding at least one additional predetermined residue to the 3′ terminus of said cDNA with a terminal deoxynucleotidyl transferase; and
said step of linking a second known segment to the 3′ terminus of said cDNA is carried out by:
(i) binding a second bridge primer to said cDNA, said second primer comprising, in the 5′ to 3′ direction, a second known segment and at least one corresponding residue, which corresponding residue binds to said at least one additional predetermined residue by Watson-Crick pairing, said second primer having an inactivated predetermined residue on the 3′ terminus thereof; and then
(ii) further transcribing said cDNA from second bridge primer by elongation of said at least one additional predetermined residue with reverse transcriptase so that a cDNA is produced having said first known segment on the 5′ terminus thereof and said second known segment on the 3′ terminus thereof.

4. The method of claim 3, wherein said at least one additional unmatched residue is selected from the group consisting of A, T, C, G, and oligomers thereof, and said at least one corresponding residue is selected from the group consisting of A, T, C, G, and oligomers thereof.

5. The method of claim 3, wherein said at least one additional unmatched residue is selected from the group consisting of C and oligo C, and said at least one corresponding residue is selected from the group consisting of G and oligo G.

6. The method of claim 1, wherein said step of linking a second known segment to the 3′ terminus of said cDNA is carried out by directly linking said second known segment to said 3′ terminus with RNA ligase.

7. The method of claim 6, wherein said second known segment comprises a DNA.

8. The method according to claim 1, further comprising the step of:
(e) amplifying said cDNA with a pair of primers, one of which pair binds to said first known segment and the other of which pair binds to said second known segment.

9. The method according to claim 8, wherein said amplifying step comprises a polymerase chain reaction amplification step.

10. The method according to claim 8, wherein said amplifying step comprises an amplification process selected from the group consisting of strand displacement amplification, rolling circle amplification, and fluorescent oligonucleotide dendrimeric signal amplification.

11. A method of uniformly amplifying a plurality of different target mRNAs in a sample, said method comprising the steps of:
(a) binding a first primer to a each of said target mRNA, the first primer comprising, in the 5′ to 3′ direction, a first known segment and an oligo T segment;
(b) transcribing a cDNA from said each of said target mRNA by elongation of said first primer with reverse transcriptase; then
(c) linking a second known segment to the 3′ terminus of each of said cDNAs; and then
(d) uniformly amplifying each of said cDNAs with a pair of primers, one of which pair binds to said first known segment and the other of which pair binds to said second known segment.

12. The method of claim 11, wherein:
said step of transcribing a cDNA from each of said target mRNA is carried out so that at least one additional unmatched C residue is produced on the 3′ terminus of said cDNAs; and
said step of linking a second known segment to the 3′ terminus of each said cDNAs is carried out by:
(i) binding a second bridge primer to each of said cDNAs, said second primer comprising, in the 5′ to 3′ direction, a second known segment and at least one G residue, said second primer having an inactivated G residue on the 3′ terminus thereof; and then
(ii) further transcribing said cDNAs from second bridge primer by elongation of said at least one additional C residue with reverse transcriptase so that a plurality of cDNAs is produced having said first known segment on the 5′ terminus thereof and said second known segment on the 3′ terminus thereof.

13. The method of claim 11, wherein:
said step of transcribing a cDNA from each of said target mRNAs is followed by the step of adding at least one additional predetermined residue to the 3′ terminus of each of said cDNAs with a terminal deoxynucleotidyl transferase; and
said step of linking a second known segment to the 3′ terminus of said cDNAs is carried out by:
(i) binding a second bridge primer to each of said cDNAs, said second primer comprising, in the 5′ to 3′ direction, a second known segment and at least one corresponding residue, which corresponding residue binds to said at least one additional predetermined residue by Watson-Crick pairing, said second primer having an inactivated residue on the 3′ terminus thereof; and then
(ii) further transcribing said cDNAs from second bridge primer by elongation of said at least one additional predetermined residue with reverse transcriptase so that a plurality of cDNAs is produced having said first known segment on the 5′ terminus thereof and said second known segment on the 3′ terminus thereof.

14. The method of claim 13, wherein said at least one additional predetermined residue is selected from the group consisting of A, T, C, G, and oligomers thereof, and said at least one corresponding residue is selected from the group consisting of A, T, C, G, and oligomers thereof.

15. The method of claim 13, wherein said at least one additional predetermined residue is selected from the group consisting of C and oligo C, and said at least one corresponding residue is selected from the group consisting of G and oligo G.

16. The method of claim 11, wherein said step of linking a second known segment to the 3′ terminus of said plurality of cDNAs is carried out by directly linking said second known segment to said 3′ terminus with RNA ligase.

17. The method of claim 16, wherein said second known segment comprises a DNA.

18. The method according to claim 11, wherein said amplifying step comprises a polymerase chain reaction amplification step.

19. The method according to claim 11, wherein said amplifying step comprises an amplification process selected from the group consisting of strand displacement amplification, rolling circle amplification and fluorescent oligonucleotide dendrimeric signal amplification.

20. The method according to claim 11, wherein said target mRNAs consists essentially of mRNA extracted from not more than 100 cells.

21. The method according to claim 11, wherein said target mRNAs consists essentially of not more than 10 nanograms of mRNA.

22. The method according to claim 11, further comprising the step of:
(e) determining the quantity of each of at least a portion of said cDNAs to thereby provide an indication of the relative amounts of the corresponding mRNAs present in said sample.

23. The method according to claim 11, wherein:
said target mRNAs consist essentially of mRNA extracted from not more than 100 cells; said method further comprising the step of:
(e) determining the quantity of each of at least a portion of said cDNAs to thereby provide an indication of the relative amounts of the corresponding mRNAs present in said sample.

24. The method according to claim 11, wherein said first known segment and said second known segment comprise the same nucleic acid sequence in opposite orientation, and wherein said amplifying step comprises a rolling circle amplification reaction.

25. A primer pair useful for amplifying an mRNA, comprising:
(a) a first primer, said first primer comprising, in the 5′ to 3′ direction, a first known segment and an oligo T segment; and
(b) a second bridge primer, said second primer comprising, in the 5′ to 3′ direction, a second known segment and a binding segment, said binding segment selected from the group consisting of A, oligo A, T, oligo T, C, oligo C, G, and oligo G, said second primer having an inactivated residue on the 3′ terminus thereof.

26. The primer pair according to claim 25, wherein said binding segment comprises an oligo C segment.

27. A kit comprising a primer pair according to claim 25.
